(19) United States
(12) Patent Application Publication
(10) Pub. No.: US 2011/0014676 A1
(43) Pub. Date: Jan. 20, 2011

(54) PROTEIN STABILIZATION

(75) Inventors: Ada S. Cowan, Liberty Township, OH (US); Richard S. Brody, Columbus, OH (US)

Correspondence Address: BATTELLE MEMORIAL INSTITUTE, 505 KING AVENUE, COLUMBUS, OH, 43201-2693, US

(73) Assignee: BATTELLE MEMORIAL INSTITUTE

(21) Appl. No.: 12/667,070

(22) PCT Filed: Jun. 27, 2008
(86) PCT No.: PCT/US08/68581

§ 371 Date: Mar. 1, 2010

Related U.S. Application Data
(60) Provisional application No. 60/937,742, filed on Jun. 29, 2007.

(51) Int. Cl. C12N 9/96; C07K 14/00; C07K 16/00

(52) U.S. Cl. 435/188; 530/350; 530/387.1; 530/388.1; 530/387.3

(57) ABSTRACT

A method and formulation for temperature stabilization of proteins, such as antibodies, enzymes such as Taq poly-merase, restriction enzymes, and other diagnostic or therapeutic enzymes using a combination of first and second stabilizers.

This invention was made with government support under Contract No. W81XWH-05-C-0078 awarded by the US Army Medical Research and Material Command. The United States Government has certain rights in this invention.

### FIELD OF THE INVENTION

The present invention relates to a method and formulation for temperature stabilization of proteins such as restriction enzymes, e.g., Taq polymerase, antibodies and other diagnostic or therapeutic proteins.

### BACKGROUND OF THE INVENTION

There is a need to stabilize therapeutic and diagnostic proteins against changes of temperature during use and storage. Currently, proteins are utilized in a variety of diagnostic and therapeutic applications. For example, one protein used in a diagnostic application is the enzyme glucose oxidase, which is used in glucose assays. The hormone insulin is an example of a protein utilized in therapeutic applications. However, proteins are particularly sensitive to certain environmental conditions and may not be stable at elevated temperatures, including physiological temperature of 37° C., in non-optimal aqueous solvent systems, or in organic solvent systems. Protein stability may also be affected by pH and buffer conditions and exposure to shear forces or other physical forces.

The stability of a protein refers to both its conformational stability, which is reflected in the protein's three-dimensional structure, and its chemical stability, which refers to the chemical composition of the protein's constituent amino acids. Protein instability can result in a marked decrease or complete loss of a protein's biological activity. Deleterious stresses such as organic solvents, interfaces between organic and aqueous solvents, extremes of pH, high temperatures, and/or dehydration (drying) can affect both the conformational and chemical stability of a protein. Chemical instability can result from processes such as (a) deamidation of the amino acids residues asparagine or glutamine, (b) oxidation of cysteine or methionine amino acid residues in the protein or (c) cleavage at any of the peptide amide linkages of the protein. Examples of conformational instability include aggregation (fibrillation), precipitation, and subunit dissociation. For reviews of protein stability see Arakawa et al., Advanced Drug Delivery Reviews, 46, 307-326 (2001) and Wang, International Journal of Pharmaceutics, 185, 129-188 (1999).

Because an inactive protein is useless, and in some cases deleterious, for most diagnostic and therapeutic applications, there is a need for a means by which proteins can be stabilized in solution at elevated temperatures (e.g. at and above room temperature, at body temperature or higher).

### BRIEF DESCRIPTION OF THE INVENTION

Broadly, the present invention is directed to a method for temperature stabilization of protein solutions or gels as well as formulations containing stabilized proteins as the active agent. The protein in solution or as a gel is stabilized by a unique stabilization system comprising a combination of a first stabilizer selected from the group consisting of an amino acid, peptide, polypeptide or poly(amino acid); and a second stabilizer selected from the group consisting of a surfactant; a monosaccharide; a disaccharide; an inorganic salt; ectoine; and combinations thereof. The unique stabilization system of the invention provides stabilization of a protein to a much greater degree of thermal stabilization than can be obtained with either stabilizer separately.

One embodiment of the present invention is directed to temperature stable aqueous solutions and gels of biologically active proteins wherein the active protein solutions and gels are stabilized by mixtures of (i) a first stabilizer based on an amino acid based compound and (ii) a second stabilizer based on a surfactant; a monosaccharide; a disaccharide; an inorganic salt; ectoine; and combinations thereof. The stable protein solutions and gels may be used in drug delivery systems and are protected against stresses such as high temperatures, oxidation, organic solvents, extremes of pH, drying, freezing, and agitation.

According to a preferred embodiment, the aqueous solutions or gels of the invention include at least one biologically active protein, wherein the protein may be an enzyme (e.g. Taq) or an antibody (e.g. anti-Yersinia pestis antibody) and at least two stabilizers for stabilizing the protein, wherein the first stabilizer is typically at least one amino acid based compound, wherein the amino acid based compound, for example, may be a protein, a peptide, a polypeptide, or a poly(amino acid). Currently preferred amino acid based stabilizers are polyarginine, oligo(arginine), arginine; poly(glutamic acid), oligo(glutamic acid), or glutamic acid. The second stabilizer is typically a small solute, a detergent, a monosaccharide, a disaccharide, a salt, or a polyionic compound.

### BRIEF DESCRIPTION OF THE DRAWINGS

FIG. 1 is a graph illustrating stabilization data at 70° C. for several Taq polymerase stabilizing formulations.

### DETAILED DESCRIPTION OF THE INVENTION AND BEST MODE

In a broad embodiment, the invention is directed to a method of preparing a temperature stabilized solution or gel of a protein in need of stabilization which comprises combining the protein to be stabilized with a stabilizing effective amount of a first stabilizer and a stabilizing effective amount of a second stabilizer;

wherein the first stabilizer is selected from the group consisting of:
a) an amino acid;
b) a peptide;
c) a polypeptide; and
d) a poly(amino acid); and
   wherein said second stabilizer is selected from the group consisting of:
a) a surfactant;
b) a monosaccharide
c) a disaccharide;
d) an inorganic salt;
e) ectoine;
f) a polyionic compound; and
g) one or more of an amino acid, peptide, polypeptide, poly(amino acid); provided that such amino acid, peptide, polypeptide, or poly(amino acid) is not selected as a first stabilizer; and
h) combinations of any of components a-g of said second stabilizer group.

Yet another broad embodiment of the invention is directed to a formulation containing a temperature stabilized solution or gel of a protein in need of stabilization which formulation comprises a combination of said protein and a stabilizing effective amount of a first stabilizer and a stabilizing effective amount of a second stabilizer;

wherein the first stabilizer is selected from the group consisting of:
a) an amino acid;
b) a peptide;
C\ a polypeptide; and
d) poly(amino acid); and
   wherein said second stabilizer is selected from the group consisting of:
a) one or more of a surfactant;
b) one or more of a monosaccharide
c) one or more of a disaccharide;
d) one or more of an inorganic salt;
e) ectoine;
f) one or more of a polyionic compound; and
g) one or more of an amino acid, peptide, polypeptide, poly(amino acid); provided that such amino acid, peptide, polypeptide, or poly(amino acid) is not selected as a first stabilizer; and
h) combinations of any of components a-g of said second stabilizer group.

Another embodiment of the invention is directed to a method of preparing a temperature stabilized aqueous solution or gel of Taq polymerase which comprises combining said Taq polymerase with:
(i) a stabilizing effective amount of a first stabilizer, wherein said first stabilizer is selected from the group consisting of:
   a) a basic amino acid;
   b) an acidic amino acid;
   c) an acidic or a basic poly(amino acid); and
(ii) a second stabilizer selected from the group consisting of:
   a) a surfactant;
   b) a monosaccharide or a disaccharide;
   c) one or more of an inorganic salt;
   d) ectoine;
   e) combinations of any of components a)-c) of said first stabilizer group provided that such basic amino acid, acidic amino acid, or said acidic or basic poly(amino acid) is not selected as said first stabilizer; and
   f) combinations of any of components a)-e) of said second stabilizer group.

Yet another embodiment of the invention is directed to a temperature stabilized formulation of Taq polymerase which is an aqueous solution or gel, containing a stabilizing effective amount of:
(i) a first stabilizer selected from the group consisting of:
   a) a basic amino acid;
   b) an acidic amino acid; and
   c) an acidic or basic poly(amino acid); and
(ii) a second stabilizer selected from the group consisting of:
   a) a surfactant;
   b) a monosaccharide or a disaccharide;
   c) an inorganic salt; and
   d) ectoine;
   e) combinations of any of components a-c of said first stabilizer group provided that such basic amino acid, acidic amino acid, or said acidic or basic poly(amino acid) is not selected as said first stabilizer; and
   f) combinations of any of components a-e of said second stabilizer group.

Biologically Active Proteins. The term, “protein” as used herein is used according to its generally under stood meaning and refers to macromolecules that are constructed from one or more unbranched chains of amino acids. A typical protein contains 200-300 amino acids. As used herein, the term “protein” specifically refers to biologically active polymerases, restriction enzymes, antibodies, diagnostic proteins and therapeutic proteins.

The term “polymerase” as used herein refers to an enzyme whose central function is associated with polymers of nucleic acids such as RNA and DNA. The primary function of a polymerase is the polymerization of new DNA or RNA against an existing DNA or RNA template in the processes of replication and transcription. In association with a cluster of other enzymes and proteins, they take nucleotides from solution, and catalyze the synthesis of a polynucleotide sequence against a nucleotide template strand using base-pairing interactions.

A DNA polymerase is an enzyme that assists in DNA replication. Such enzymes catalyze the polymerization of deoxyribonucleotides alongside a DNA strand, which they “read” and use as a template. The newly-polymerized molecule is complementary to the template strand and identical to the template's partner strand.

A RNA polymerase produces a transcription unit that extends from the promoter to the termination sequences. The gene is defined in reference to the start site—those sequences before the start site are called the upstream sequences, those after the start site are called downstream sequences. The immediate product is the primary transcript.

As would be recognized by one skilled in the art, various polymerases are commercially available from suppliers that readily may be found by doing an internet search. One well known supplier is New England Biolabs, 240 County Road, Ipswich, Mass. 01938-2723 USA. New England Biolabs supplies the following types of polymerases: PreCR, PCR Products, qPCR Products. RT-PCR and qRT-PCR, Amplification and Cloning Technologies, Thermophilic DNA Polymerases, Mesophilic DNA Polymerases, Reverse Transcriptases, and RNA Polymerases.

The preferred polymerase for use in the formulations and method of the invention is Taq polymerase. It is often abbreviated to “Taq Pol” (or simply “Taq”), and is frequently used in polymerase chain reaction (PCR) methods for greatly amplifying short segments of DNA. New England Biolabs has eight (8) various Taq products available.

The Term “restriction enzyme” as used herein, refers to enzymes which are DNA-cutting enzymes found in bacteria (and harvested from them for use). Because restriction enzymes cut within the molecule, they are often called restriction endonucleases. These restriction enzymes are readily commercially available and over 100 restriction enzymes are available from New England Biolabs.

A restriction enzyme recognizes and cuts DNA only at a particular sequence of nucleotides. For example, the bacterium Hemophilus aegypticus produces an enzyme named HaeIII that cuts DNA wherever it encounters the sequence

5′GGCC3′
3′CCGG5′.

The cut is made between the adjacent G and C. This particular sequence occurs at 11 places in the circular DNA molecule of the virus phiX174. Thus, treatment of this DNA with the enzyme produces 11 fragments, each with a precise length and nucleotide sequence.

Other restriction enzymes that may be useful in the methods and formulations of the invention include Ava I, Bam HI, BgI II, Eco RI, Eco RII, Eco RV, Hha I, Hind III, Hpa I, Kpn I, Mbo I, Pot I, Sma I, Sstl, Sal I, Taq I, and Xma I.

As used herein, the term “antibody” or “antibodies” also known as immunoglobulins) are gamma globulin proteins that are found in blood or other bodily fluids of vertebrates, and are used by the immune system to identify and neutralize foreign objects, such as bacteria and viruses. They are typically made of basic structural units—each with two large heavy chains and two small light chains—to form, for example, monomers with one unit, dimers with two units or pentamers with five units. Antibodies are produced by a kind of white blood cell called a B cell.

Antibodies that have been stabilized by the methods and formulations of the invention include anti-Yp monoclonal antibody, goat anti-Yp polyclonal antibody, rabbit anti-ricin antibody, and rabbit anti-ovalbumin antibody.

The term “diagnostic proteins” as used herein, refers to diagnostic proteins used to detect certain diseases in humans and animals. The following examples are illustrative of, but not limited to diagnostic proteins known in the art: 31 kD proteins from Schistosoma mansoni worms; purified proteins analyzed using sera from rabbits immunized with M. paratuberculosis which causes Johne's disease in cattle; polyamine-modified Aβ40 used to target Alzheimer's amyloid plaques, and A-Protein used as a diagnostic of cancer.

The term “therapeutic proteins” is used herein to refer to proteins that are engineered in the laboratory for pharmaceutical use. The majority of biopharmaceuticals marketed to date are recombinant therapeutic protein drugs. Examples of therapeutic proteins are exemplified by but not limited to Pegasys (peginterferon alpha 2-a) for treatment of hepatitis C and hepatitis B; Humira® (adalimumab) for treatment of rheumatoid arthritis and Crohn's disease; Fabrazyme® (agalsidase beta) for treatment of Fabry's Disease; Amevive® (alefacept) for treatment of psoriasis; Herceptin® (trastuzumab) for treatment of breast cancer; Aranesep® (darbepoetin) for treatment of anemia; Remicade® (infliximab) for treatment of rheumatoid arthritis and Crohn's disease; Rituxan® (rituxamab) for treatment of rheumatoid arthritis and non-Hodgkin's lymphoma; Bexxar® (tositumomab) for treatment of non-Hodgkin's lymphoma; Avastin® (bevacizumab) for treatment of metastatic colorectal cancer; and Erbitux® (cetuximab) for treatment of metastatic colorectal cancer.

The biologically active proteins used in the methods and formulations of the invention will be present in the solution or gel at an amount effective to accomplish the function of the protein. For example, if a therapeutic protein such as peginterferon alpha 2-a is stabilized according to the methods of the invention it will be present in the solution at an amount effective to treat hepatitis C or B in accordance with its approved package insert. As used herein, the term “approved” refers to approval to market a drug by the U.S. Food and Drug Administration (FDA) or other international approval bodies.

The stabilized formulations of the invention are solutions or gels and may be prepared using aqueous or organic solvents. Since proteins are generally soluble in water, and since water is a benign material, the use of an aqueous solvent is preferred herein; however, under some circumstances it may be necessary to use a mixture of aqueous and an organic solvent such as ethanol to form the solutions or gels of the invention.

The formulations of the invention are stabilized against extremes of temperature by a unique combination of a first group and second group of stabilizing agents (Group 1 and Group Stabilizers) which are described in detail below.

Group 1 Stabilizers.

The term “amino acid” as used herein means the stereoisomeric forms, e.g. D and L forms, the following compounds: alanine, β-alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine, γ-aminobutyrate, Nε-acetyllysine, Nδ-acetylornithine, Nγ-acetyldiaminobutyrate and Nα-acetyldiaminobutyrate. L-amino acids are preferred. Particularly preferred amino acids for use herein are arginine and glutamic acid or mixtures of arginine and glutamic acid preferably 1:1 mixtures.

Basic amino acids are polar and positively charged at pH values below their pKₐ's, and are very hydrophilic; histidine, lysine and arginine are basic amino acids. Acidic amino acids are negatively charged, polar and hydrophilic and include aspartic acid and glutamic acid. The amino acid will be present in the stabilized formulations of the invention at from about 1% to about 40%, preferably from about 5% to about 30% by weight.

The term “peptide” encompasses a sequence of two or more amino acids wherein the amino acids are naturally occurring or synthetic (non-naturally occurring) amino acids. The term “peptide” typically refers to short polypeptides. Typically, a peptide may be used as a Group 1 stabilizer at a concentration at from about 1% to about 30% by weight.

The term “polypeptide” as used in this application refers to a polymer composed of amino acid residues, related naturally occurring structural variants, and synthetic non-naturally occurring analogs thereof linked via peptide bonds, and synthetic non-naturally occurring analogs thereof. Synthetic polypeptides can be synthesized, for example, using an automated polypeptide synthesizer. If a polypeptide is used as the Group 1 stabilizer it may be used at a concentration of from about 1% to about 30%, preferably from about 1% to 20% by weight.

The term “polyamino acid” as used herein refers to a synthetic polymer made up of many repeating units of amino acid(s). A homo polyamino acid is a polymer made up of a single amino acid as the repeating unit. A random co-polyamino acid is a polymer made from two or more different amino acids that repeat in a random sequence. Natural poly(amino acid)s are a group of poly(ionic) molecules (ionomers) with various biological functions. Preferred for use herein in the methods and formulations of the invention are poly/arginine), oligo(arginine) and poly(glutamic acid). If poly(amino acids) are selected as the Group 1 stabilize the will be present in the formulation at a concentration of from about 0.1% to about 15%, preferably from about 0.3% to about 5% by weight.

Group 2 Stabilizers

The terms “surfactants” or “detergents” are used herein to refer to wetting agents that lower the surface tension of a liquid, allowing easier spreading, and lower the interfacial tension between two liquids. Surfactants are usually organic compounds that are amphiphilic, meaning they contain both hydrophobic groups (their “tails”) and hydrophilic groups (their “heads”). Therefore, they are soluble in both organic solvents and water. Preferred surface active agents (surfactants) include nonionic and ionic surfactants. Two or more surface modifiers can be used in combination.

Although gelatin and glycerol are not typically used as surfactants, they both have surface modifying properties. Gelatin is commonly used as an emulsifier and glycerol is commonly used as a humectant and thickening agent; accordingly, they are placed in the category of surfactant for use as a Group Stabilizer.

Representative examples of surface modifiers that may be useful in the unique stabilizing system of the invention include gelatins, glycerol, dipalmitoylphosphatidylcholine, sorbitan esters; polyoxyethylene alkyl ethers (e.g., macrogol ethers such as Cetomacrogol 1000); polyoxyethylene castor oil derivatives; polyoxyethylene sorbitan fatty acid esters (e.g., the commercially available Tweens® such as e.g., Tween 20® and Tween 80® from ICI Specialty Chemicals; polyethylene glycols (e.g., Carbowaxs 3350® and 1450®, and Carbopol 934® from Union Carbide; dodecyl trimethyl ammonium bromide; polyoxyethylene stearates; sodium dodecylsulfate; triethanolamine; polyvinyl alcohol (PVA); polyvinylpyrrolidone (PVP); 4-(1,1,3,3-tetramethylbutyl)-phenol polymer with ethylene oxide and formaldehyde (also known as tyloxapol); poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine from BASF Wyandotte Corporation, Parsippany, N.J.; a charged phospholipid such as dimyristoyl phophatidyl glycerol, dioctylsulfosuccinate (DOSS); alkyl aryl polyether sulfonate; decanoyl-N-methylglucamide; n-decyl β-D-glucopyranoside; n-decylp β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like.

The surfactants listed above are known in the art and are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference. The surface modifiers are commercially available and/or can be prepared by techniques known in the art.

Applicants have found that dipalmitoylphosphatidylcholine (DPPC), gelatin, glycerol, Tween® 80 (polyoxyethylene (20) sorbitan monooleate), Tween® 20 (polyoxyethylene (20) sorbitan monolaurate), Pluronic® F68 (polyoxyethylene-polyoxypropylene block copolymer) and Brij-35® (polyoxyethyleneglycol dodecyl ether) are especially useful in the methods and formulations of the invention.

In general, if a surfactant is selected as the Group Stabilizer it will be present in the formulations at from about 0.01% to about 10%; preferably about 0.1% to about 5%. When a surfactant is selected as a stabilizer, one important aspect of the invention is that the formulations have a concentration below the critical micelle concentration. Critical micelle concentration is defined as that above which micelles form.

As used herein, the term “monosaccharide” refers to the simplest carbohydrates which cannot be hydrolyzed into simpler sugars. They consist of one sugar and are usually colorless, water-soluble, crystalline solids. Monosaccharides such as pentoses and hexoses are useful herein. The pentoses include arabinose, lyxose, ribose, xylose, ribulose, xylulose, and mixtures thereof. The hexose maybe an aldohexose including allose, altrose, galactose, glucose, gulose, idose, mannose, and talose, or ketohexoses including fructose, psicose, sorbose, tagatose, and mixtures thereof. The monosaccharides preferred for use herein include glucose (dextrose), fructose, galactose, xylose and ribose. If a monosaccharide is selected for use as a Group 2 Stabilizer it will be present in the formulation at from about 15% to about 20% and preferably at from about 5% to about 10%.

The term “disaccharide” refers to a sugar (a carbohydrate) composed of two monosaccharides. Disaccharide is one of the four chemical groupings of carbohydrates (monosaccharide, disaccharide, oligosaccharide, and polysaccharide). The commonly used disaccharides are listed in the following Table A.

TABLE A
List of Disaccharides

| Disaccharide | Unit 1 | Unit 2 | Bond |
|---|---|---|---|
| Sucrose (table sugar, cane sugar, | glucose | fructose | α(1→2) |
| saccharose, or beet sugar) | | | |
| Lactose (milk sugar) | galactose | glucose | β(1→4) |
| Maltose | glucose | glucose | α(1→4) |
| Trehalose | glucose | glucose | α(1→1)α |
| Cellobiose | glucose | glucose | β(1→4) |

Less common disaccharides include: gentiobiose, that consists of two glucose monomers with an β(1→6) linkage; isomaltose, that consists of two glucose monomers with an α(1→6) linkage; Kojibiose, that consists of two glucose monomers with an α(1→2) linkage; laminaribiose, that consists of two glucose monomers with a β(1→3) linkage; mannobiose, that consists of two mannose monomers with either an α(1→2), α(1→3), α(1→4), or an α(1→6) linkage; melibiose, that consists of a glucose monomer and a galactose monomer with an α(1→6) linkage; nigerose, that consists of two glucose monomers with an α(1→3) linkage; rutinose, that consists of a rhamnose monomer and a glucose monomer with an α(1→6) linkage; and xylobiose, that consists of two xylopyranose monomers with a β(1→4) linkage.

The preferred disaccharides for use herein include sucrose and trehalose; especially preferred for use in the stabilized formulations of the invention is trehalose. The disaccharide will generally be present in the formulations of the invention at from about 1% to about 40% and preferable from about 30% to 40%.

As used herein, the term “inorganic salt” as used herein include salts of for example, bicarbonate, borate, bromide, carbonate, chloride, chlorite, fluoride, hydrosulfite, iodide, molybdate, nitrate, persulfate, phosphate, sulfate and thiosulfate. Any inorganic salt that is approved for use in humans and animals and which is pharmaceutically acceptable may be used herein provided the salt has no deleterious effect on the protein active agent. Preferred salts for use herein are sodium chloride, sodium phosphate, potassium phosphate, lithium chloride, calcium chloride, sodium sulfate and the like. The inorganic salt will preferably be present in the solutions of the invention at from about 100 mM to about 1 M.

Ectoine is a natural compound which serves as a protective substance in many bacterial cells and is a preferred Group 2 Stabilizer. Ectoine and the ectoine derivatives are low molecular weight cyclic amino acid derivatives which can be obtained from various halophilic microorganisms and confers resistance towards salt and temperature stress. Ectoine was first identified in the microorganism Ectothiorhodospira halochloris, but has since been found in a wide range of gram-negative and gram-positive bacteria. The chemical name of ectoine is (4S)-2-methyl-3,4,5,6-tetrahydropyrimidine-4-carboxylic acid (C₆H₁₀N₂O₂) and hydroxyectoine is (S,S)-1,4,5,6-tetrahydro-5-hydroxy-2-methyl-4-pyrimidinecarboxylic acid. In general, ectoine will be present in the solutions of the invention at from about 10% to about 40%, preferably from about 20% to about 40% and preferably at about 30% by weight.

Preferred “polyionic compounds” or “polyions” for use in the present invention include polyamino acids, e.g., proteins, polypeptides, i.e., polylysine, polyhistidine, and polyarginine. Other polyions that are useful in accordance with the invention include organic polyions, i.e., polyacrylic acid, polycarboxylic acids, polyamines (e.g., polyethylamine), polysulfonic acids (e.g., polystyrene sulfonic acid) polyphosphoric acid (e.g., polyvinylphosphoric acid), or copolymers of any or all of these, e.g., mixed polymers of these polyamino acids, and the like. Typically, the polyion will range in size from about 5 kD to about 1000 kD, and preferably, from about 10 kD to about 100 kD. In the case of polyamino acids, this typically constitutes a polymer of from about 50 to about 10,000 amino acid monomers in length, and preferably from about 100 to about 1000 monomers in length. Preferred polyionic compounds for use herein are dextran sulphates, poly-L-lysines, and polyethleneimine; especially preferred for use herein is polyethleneimine (PEI) or polyacrylic acid (PAA) at from about 0.05% to about 1% by weight.

Broadly, it has been discovered that the combination of a Group 1 Stabilizer with the Group 2 Stabilizers such as those presented in Table B provides for protein stabilization at reduced amounts of stabilizer materials. The present invention provides methods and formulations for stabilizing proteins, e.g., enzymes such as Taq polymerase, restriction enzymes, and other diagnostic or therapeutic enzymes.

Although It is contemplated herein that a single Group 1 Stabilizer may be used in the liquid or gel formulations of the invention, it is also contemplated to use combinations or mixtures of Group 1 Stabilizers as for example a combination of arginine and glutamic acid at 2% by weight of each amino acid. It is also contemplated that Group 2 Stabilizers may be combined to produce an optimal stabilization combination. For example, the Group 2 Stabilizer may be a combination of trehalose and an arginine/glutamic acid mixture, or a combination of trehalose and NaCl or CaCl₂. It is within the skill of the art to select the optimal combination of Group 1 and Group 2 Stabilizers to effectively stabilize a particular protein.

Unless other wise provided herein percent “%” refers to weight %. Also, as used herein, Tables with alpha designators, e.g., A, B. C, etc. contain informational material. Tables with numeric designators e.g., 1, 2, 3, etc., contain summarized data.

Table B below lists typical Group 1 and Group 2 Stabilizers and their concentration useful in the stabilizing system of the invention.

TABLE B
Typical First and Second Stabilizers

| FIRST | SECOND | |
|---|---|---|
| STABILIZERS | STABILIZERS | TYPICAL STABILIZERS |
| | Monosaccharides | Trehalose (Tre; 5-40%) |
| | disaccharides | Sucrose (30%) |
| | Salts | Sodium Chloride (0.15 M-2 M) |
| | | Magnesium Chloride (0.1 M) |
| | | Sodium Sulfate (1-2 M) |
| | | Potassium Phosphate (KPi; 1-3 M) |
| | Small Solutes | Ectoine (1,4,5,6-Tetrahydro-2- |
| | | methyl-4-pyrimidinecarboxylic |
| | | acid; 20-30%) |
| | Surfactants | Tween 20 ® and 80 ® (T20, |
| | (Detergents) | T80; 0.01-1%) |
| | | Pluronic F68 ® (0.1-1%) |
| | | Brij 35 ® (0.1-1%) |
| | | Octyl-glucopyranoside (C8GP; |
| | | 0.1-2%) |
| | | Palmitic Acid (0.1-1%) |
| | | Dipalmitylphosphatidylcholine |
| | | (DPPC; 1-1%) |
| | | Hydroxypropl-β-Cyclodextrein |
| | | (HPCD; 1-8%) |
| | Polycations | Polyethyleneimine (PEI: 0.05-1%) |
| | Polyanions | Polyacrylic Acid (PAA: 0.1-1%) |
| | Surfactant | Gelatin A (Gel A; 0.1-4%) |
| | | Gelatin B (Gel B; 0.1-4%) |
| Amino Acids | Amino Acid | Arginine (Arg; 5-30%) |
| Based* | Based* | Arginine/Glutamic Acid |
| | | (Arg/Glu; 2% each |
| | | Poly (Glutamic acid) |

*Amino acid based compounds may be both first and secondary stabilizers as long as a different amino acid based compound is selected for each.

Multiple effective stabilizing formulations were developed at room temperature, 40° C., and 70° C. The disclosed formulations allow the storage of Taq polymerase at ambient temperatures for up to nine months to one year, removing the need for refrigerated storage and increasing the reagent's ease of use in the field or laboratory.

Preferably the aqueous solution of the protein to be stabilized is prepared with conventional buffering solutions known in the art as for example pH 7.2 phosphate buffered saline (PBS). To prepare PBS, stock solutions are prepared of monobasic sodium phosphate 0.2M by adding 27.6 g/liter of dH₂O; a stock solution of dibasic sodium phosphate (anhydrous) 0.2 M is prepared by adding 28.4 g/liter dH₂O. A quantity of 7.2 pH buffer is prepared as shown in the following chart:

| | |
|---|---|
| 500 ml | 1000 ml |

| | | | | |
|---|---|---|---|---|
| Monobasic | 140 | ml | 280 | ml |
| Sodium phosphate | | | | |
| Dibasic sodium | 360 | ml | 720 | ml |
| Phosphate (anhydrous) | | | | |
| NaCl | 4.5 | g | 9 | g |

The stabilized protein solution is thereafter filled into sterile vials and sealed using conventional pharmaceutical filling and capping equipment and are either stored frozen or at 4° C. until used for the purpose the protein is normally used. Alternatively, the vials are lyophilized and the lyophilized vials are held at 4° C. or at controlled room temperature until reconstituted with sterile water, or saline solution.

One embodiment of the invention provides for a method and formulations that stabilize liquid proteins containing an enzyme such as Taq polymerase. The formulations are designed to eliminate the need for specialized storage conditions for liquid protein formulations, as well as to extend the shelf life for these intrinsically temperature sensitive reagents.

The benefit of stabilized protein reagents for portable assays is clear, as it may not be possible to regulate storage and assay temperatures in varying environments. In addition, stabilized protein reagents will benefit laboratory assays by increasing the reliability of the reagents before, during and after shipping, storage on-site, and during use of the assay itself. This is especially important for high throughput screening where liquid reagents may need to remain at room temperature for several days in a queue. There is also a strong likelihood that the amount of reagent that needs to be shipped from the reagent manufacturer to end users can be dramatically reduced, which creates another source of benefit via reduced product cost.

In order to demonstrate the effectiveness of the methods and compositions of the invention, the following assay was used to quantitate Taq polymerase activity and illustrates the effectiveness of the stabilization method of the invention.

### Assay to Quantitate Taq Activity

An activity assay Of Tan polymerase was used using a one cycle PCR reaction system where the activity of Taq polymerase was directly quantified by measuring the rate of incorporation of ³²P-labeled dCTP into nicked DNA.

The one cycle reaction system involved reagents and conditions similar to those used in normal PCR, except for the addition of ³²P-labeled dCTP and the use of a single reaction temperature (74° C. for 10-20 minutes). The synthesized ³²P-labeled DNA was precipitated with trichloroacetic acid (TCA) and unincorporated free ³²P-labeled dCTP was washed from the precipitated ³²P-labeled DNA.

Unit activity calculations were performed by converting the ³²P count to the amount of incorporated nucleotides. One unit of Taq polymerase activity is the amount of polymerase required to incorporate 10 nmol of deoxynucleoside triphosphate into acid-insoluble at 74° C. in 30 minutes under standard conditions.

The original Taq polymerase activity assay using ³²P obtained from the vendor was not a high throughput procedure (using a single filter reaction). An in house 96 well assay format was used to screen multiple stabilizers in a highly efficient fashion.

In the stabilization tests, the absolute enzyme activity is not necessary. The relative rates of ³²P incorporation into synthesized DNA catalyzed by Taq polymerase that had been stored under different conditions were used.

Taq DNA polymerase (400-600 U/μL) was obtained from Invitrogen. This thermostable enzyme can withstand prolonged incubation at temperatures up to 95° C. without significant loss of activity for a few hours. The enzyme consists of a single polypeptide with a molecular weight of 94 kDa. It has a 5′ a3′ DNA polymerase activity and a 5′ a3′ exonuclease activity.

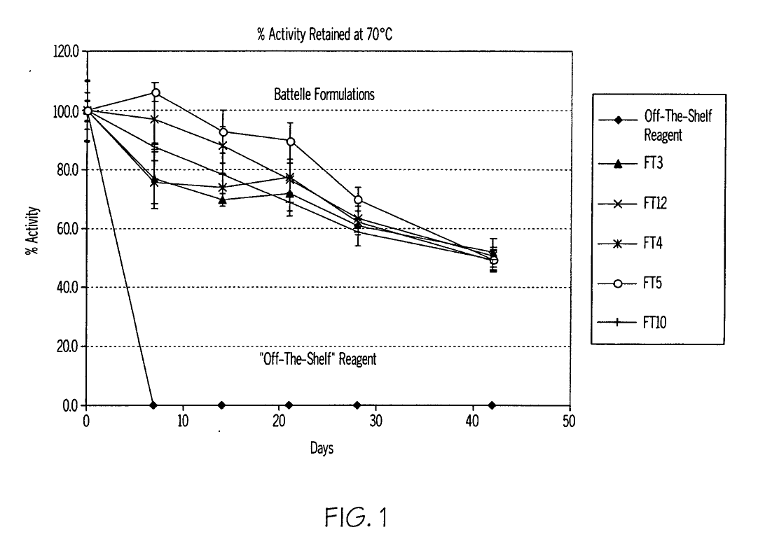

Referring now to FIG. 1, this figure is a graph that shows stabilization data for Taq at 70° C. The off-the-shelf reagent lost all activity within a few days while stabilized formulations according to the invention maintained more than 90% and 50% of activity at 70° C. after two weeks and six weeks storage, respectively.

The abbreviations listed in Table C were used in the Tables 1-9.

TABLE C
List of Abbreviations

| Abbreviation | Purpose or Action | Full Name |
|---|---|---|
| Arg | Amino Acid | Arginine |
| DPPC | Surfactant | Dipalmitoylphosphatidylcholine |
| Ecto | 2^{nd} Stabilizer | Ectoine |
| PluF68 | Surfactant | Pluronic ® F68 |
| PolyGlu | Polyamino acid | Poly(glutamic acid) |
| T20 ® | Surfactant | Tween ® 20 |
| T80 ® | Surfactant | Tween ® 80 |
| Glu | Amino Acid | Glutamic acid |
| Tre | Disaccharide | Trehalose |
| N.D. | | Not Determined |

As illustrated by Table 1, which shows Taq polymerase stabilization data at ambient temperature and 40° C. Activity as high as 90% and 75% was maintained at ambient temperature and 40° C., respectively, after nine months of storage. The off-the-shelf reagent lost all activity within 10 days.

TABLE 1
% Taq Activity Retained After Stabilization

| | FORMULATION | | | 25° C. | 40° C. | 70° C. | |
|---|---|---|---|---|---|---|---|
| | | | Second | After 9 | | After 3 | After 6 |
| SAMPLE # | Taq | First Stabilizer | Stabilizer | Months | | Weeks | Weeks |
| Control | yes | 0 | 0 | 0 | 0 | 0 | 0 |
| FT3 | yes | Arginine 30% | 0 | 88 | 49 | 72 | 52 |
| FT4 | yes | Arginine | Plu F68 ® | 69 | 41 | 77 | 51 |
| | | (20%) | (0.1%) | | | | |
| FT5 | yes | Arginine | DPPC | 47 | 21 | 90 | 49 |
| | | (20%) | (0.1%) | | | | |
| FT10 | yes | Arginine | NaCl (1M) | 74 | 52 | 69 | 50 |
| | | (20%) | | | | | |
| FT11 | yes | Arginine | Trehalose | 85 | 74 | 62 | 20 |
| | | (20%) | (10%) | | | | |
| FT12 | yes | Arginine | Ectoin | 91 | 62 | 77 | 49 |
| | | (20%) | (20%) | | | | |
| FT15 | yes | Poly(glutamic | T20 ® | 69 | 37 | ND | ND |
| | | acid) (0.03%) | (0.5%) | | | | |

Table 2 illustrates the Taq stabilization data for 70° C. As can be seen a small amount of second stabilizer allowed a 33 percent reduction in first stabilizer concentration (arginine) content while maintaining similar or better activity retention. Particularly good results were obtained with secondary stabilizers such as 1M NaCl, 0.1% Plu F68®, and 0.1% DPPC.

Table 3 illustrates the combination of 20% Arginine with various concentrations of secondary stabilizer. The table shows that the combination of 20% Arginine with the secondary stabilizers gave much better results than the secondary stabilizer alone.

Table 4 illustrates first and secondary stabilizer data for Taq at 70° C. A 33% reduction in Arginine content plus a small amount of secondary stabilizer gave equal or better stability.

TABLE 2
% Activity in Taq Stabilization Maintained After
Indicated Time at 70° C.

| | | 2^{nd} | Time | Time | Time | Time |
|---|---|---|---|---|---|---|
| Sample No. | 1^{st} Stabilizer | Stabilizer | 2 wks | 3 wks | 4 wks | 6 wks |
| A1-3 | 30% Arg | 0 | 60 | 72 | 67 | 52 |
| A2-2 | 20% Arg | 0 | 83 | 44 | 38 | ND |
| A3-2 | 5% Arg | 0 | 77 | 32 | 23 | ND |
| A4-3 | 1% Arg | 0 | 26 | 11 | 6 | 2 |
| A5-3 | 20% Arg | 20% | 74 | 77 | 62 | 49 |
| | | Ectoine | | | | |
| A6-3 | 20% Arg | 10% | 57 | 62 | 38 | 20 |
| | | Trehalose | | | | |
| A7-3 | 20% Arg | 1M NaCl | 78 | 69 | 59 | 50 |
| A8-3 | 20% Arg | 0.1% Plu | 88 | 77 | 63 | 51 |
| | | F68 ® | | | | |
| A9-3 | 20% Arg | 0.1% | 93 | 90 | 70 | 49 |
| | | DPPC | | | | |

TABLE 3
Taq % Activity Maintained After Indicated Time @ 70°

| | | 2^{nd} | 1 | 2 | 3 | 4 | 6 |
|---|---|---|---|---|---|---|---|
| Sample # | 1^{st} Stabilizer | Stabilizer | week | weeks | weeks | weeks | weeks |
| B1-1 | 0 | 40% Ectoine | 39 | ND | 37 | 33 | ND |
| B2-3 | 0 | 40% Ectoine | 40 | 29 | 20 | 10 | ND |
| B3-2 | 0 | 30% Ectoine | 61 | 32 | ND | ND | ND |
| B4-2 | 0 | 20% Ectoine | 43 | ND | 23 | 11 | ND |
| B5-3 | 20% Arg | 20% Ectoine | 76 | 74 | 77 | 62 | 49 |
| Control | 0 | 0 | 0 | 0 | 0 | 0 | 0 |
| B6-1 | 0 | 30% Trehalose | 30 | 9 | ND | ND | ND |
| B7-2 | 0 | 40% Trehalose | 13 | 5 | 1 | 0 | ND |
| B8-2 | 0 | 5% Trehalose | 40 | 53 | 13 | 3 | ND |
| B9-3 | 0 | 1% Trehalose | 15 | 1 | 0 | ND | ND |
| B10-3 | 20% Arg | 10% Trehalose | 77 | 57 | 62 | 38 | 20 |
| Control | 0 | 0 | 0 | 0 | 0 | 0 | 0 |
| B11-1 | 0 | 150 mM NaCl | 33 | 20 | ND | ND | ND |
| B12-2 | 0 | 1 M NaCl | 52 | 65 | 35 | 16 | ND |
| B13-3 | 20% Arg | 1M NaCl | 88 | 78 | 69 | 59 | 50 |
| B14-1 | 0 | 1% Plu F68 ® | 31 | 20 | ND | ND | ND |
| B15-1 | 0 | 0.1% Plu F68 ® | 34 | 19 | ND | ND | ND |
| B15-A | 20% Arg | 0.1% Plu F68 ® | 97 | 88 | 77 | 63 | 51 |
| B16-1 | 0 | 1% DPPC | 39 | 23 | ND | ND | ND |
| B17-1 | 0 | 0.1% DPPC | 44 | 21 | ND | ND | ND |
| B17-A | 20% Arg | 0.1% DPPC | 106 | 93 | 90 | 70 | 49 |
| B18-3 | 0 | 0.3% Heparin | 72 | 49 | 32 | 21 | ND |
| B19-3 | 0 | 0.03% Heparin | 75 | 58 | 36 | 22 | ND |
| B19-A | 20% Arg | 0.03% Heparin | ND | ND | ND | ND | ND |
| B20-1 | 0 | 1% Tween 20 ® | 46 | 21 | ND | ND | ND |
| B21-1 | 0 | 0.1% Tween 20 ® | 57 | 27 | ND | ND | ND |
| B21-A | 20% Arg | 0.1% Tween 20 ® | ND | ND | ND | ND | ND |

ND—not determined
Control = Taq stock formulation

TABLE 4
% Activity in Stabilization of Taq DNA polymerase at 70° C.

| | | % Activity^{a} Retained After | | | | | | |
|---|---|---|---|---|---|---|---|---|
| | | Weeks | | | | | | |
| First Stabilizer | Second Stabilizer | 0 | 1 | 2 | 3 | 4 | 5 | 8 |
| Taq Stock formulation | 0 | 100 | 0 | 0 | 0 | 0 | 0 | 0 |
| No Stabilizer 0 | | | | | | | | |
| Glycerol Free Taq - | 0 | 100 | 31.3 | 3.6 | 3.6 | 0.9 | 0.0 | 0.9 |
| No Stabilizer - 0 | | | | | | | | |
| 30% Arginine | 0 | 100 | 101.5 | 81.8 | 54.7 | 48.2 | 50.4 | 30.7 |
| 20% Arginine | 0.1% DPPC | 100 | 102.4 | 87.8 | 91.1 | 51.2 | 56.1 | 43.9 |
| 20% Arginine | 1% Gelatin A | 100 | 89.8 | 81.0 | 59.1 | 64.2 | 53.3 | 34.3 |
| 20% Arginine | 1% Tween 80 ® | 100 | 86.2 | 84.5 | 68.1 | 37.9* | 50.9 | 43.1 |
| 20% Arginine | 0.3% Poly Glutamic | 100 | 81.4 | 76.1 | 56.6 | 43.4 | 43.4 | 23.0 |
| | acid | | | | | | | |
| 20% Arginine | 0 | | | 79 | | 42 | | |
| 0 | 30% Ectoine | | | 32 | | 12 | | |
| 0 | 100 mM NaCl | | | 20 | | 10 | | |
| 0 | 0.1% Tween 20 ® | | | 27 | | 7 | | |
| 0 | 0.1% Brij 35 ® | | | 30 | | 3 | | |
| 0 | 0.1% Pluronic F68 ® | | | 19 | | 7 | | |

*unreliable

The following tables illustrate the stabilization of polyclonal antibodies and monoclonal antibodies. Table 5 illustrates first and secondary stabilizer data for Anti-Yp monoclonal antibody at elevated temperatures. Table 6 illustrates high temperature stabilizer results for Goat anti-Yp polyclonal antibody using first and secondary stabilizers. Table 7 illustrates high temperature stabilization results for Rabbit Anti-Ricin antibody using first and secondary stabilizers. Table 8 illustrates high temperature stabilizer results for Rabbit anti-ovalbumin antibody using first and secondary stabilizers. Table 9 illustrates high temperature stability tests for anti-Yp monoclonal antibody using first and secondary stabilizers.

TABLE 5
% Activity in High Temperature Stabilization Tests for Anti-Yₚ Monoclonal Antibody*

| | | | Run 1 | | | | | Run 2 | | | | | Run 3 | | | |
|---|---|---|---|---|---|---|---|---|---|---|---|---|---|---|---|---|
| | | | 60° C. | | 55° C. | | | 60° C. | | 55° C. | | | 60° C. | | 60° C. | |
| Sample #. | 1^{st} Stabilizer | 2^{nd} Stabilizer | D 1 | W 1 | W 1 | W 5 | W 6 | D 1 | W 1 | W 1 | W 3 | W 4 | D1 | W 1 | D 1 | W 1 |
| Control | 0 | 0 | — | — | 10 7 | 14 17 | 26 | — | — | 36 | 8 6 | N T | — | 5 | 7 | — |
| | | | | | | | | | | 83 | | | | | | |
| BB1 | Gel A; 2% | 0 | 29 | — | 87 | 5 | | | | | | | | | | |
| BB2 | Gel B; 2% | 0 | 18 | — | 40 | 12 | | | | | | | | | | |
| BB3 | 0 | Tre 0.5M | 11 1 | 1 | 37 | 2 | | 23 | — | 43 | 11 | | | — | | |
| BB4 | 0 | Tre 1M | | | | | | 45 | — | 39 | 25 | | 64 | — | 90 | 4 |
| BB5 | 0 | Tre 1.5M | | | | | | | | | | | 61 | 13 | | |
| BB6 | 0 | HPCD 4% | 6 | — | 44 | 4 | | | | | | | | | | |
| BB7 | 0 | T80 ® 0.1% | 1 | — | | | 30 | | | | | | | | | |
| BB8 | 0 | T80 ® 0.01% | 2 | — | 58 | | 24 | | | | | | | | | |
| BB9 | 0 | PAA, 1% | — | — | 18 | | | | | | | | | | | |
| BB10 | 0 | NaCl 1M | 16 | — | 73 | 23 | | 6 | — | 68 | 68 | | | | | |
| BB11 | 0 | NaCl 2M | | | | | | 31 | — | 78 | 24 | | | | | |
| BB12 | 1M Arg | 0 | — | — | 81 | 91 | | | | | | | | | | |
| BB13 | 0 | 1M KPi | | | | | | 70 | — | 39 | 13 | | 29 | — | 87 ± 4 7 | 2 |
| BB14 | 0 | 2M KPi | | | | | | 31 | — | 6 | 54 | | 22 | — | | |
| BB15 | 0 | 1M Na₂SO₄ | | | | | | | | | | | 17 | — | | |
| BB16 | 0 | 2M Na₂SO₄ | | | | | | | | | | | 12 | — | | |
| BB17 | 0.5 M Arg | 1M Tre | | | | | | | | | | | | | 35 | |
| BB18 | 0.5M Arg/Glu | | | | | | | | | | | | | | 56 | 1 |
| BB19 | 0.5M Arg/Glu | 1M Tre | | | | | | | | | | | | | 62 | 1 |
| BB20 | 2% Gel A | 0.1% T80 ® | | | | | | — | — | 94 | 38 | | | | | |

Control - antibody in buffer

TABLE 6
% Activity in High Temperature Stabilization Tests the Goat Anti-Yp Polyclonal Antibody

| | | | Run 1 | | | | Run 2 | | | | Run 3 | | Run 4 |
|---|---|---|---|---|---|---|---|---|---|---|---|---|---|
| | 1st | 2nd | 60° C. | | 55° C. | | 60° C. | | 55° C. | | 60° C. | | 60° C. |
| Sample # | Stabilizer | Stabilizer | D 1 | W 1 | W 1 | W 5 | W 1 | W 3 | W 1 | W 3 | W 1 | W 1.6 | W 1 |
| Control | 0 | 0 | 77 | | 64 | 89 | 16/55 | 3 | 66/10/1 | 62 | 32 | 9 | 58 |
| BBP-1 | Tre 0.5M | 0 | 97 | 14 | 74 | 47 | 30 | 4 | 56 | | | | |
| BBP-2 | Tre 1.0M | 0 | | | | | 31 | 10 | 56 | | 44 | | 68 |
| BBP-3 | Tre 1.4M | 0 | | | | | | | | | 47 | | |
| BBP-4 | HPCD 1% | 0 | | | | | 15 | | 69 | 63 | | | |
| BBP-5 | HPCD 4% | 0 | 97 | 10 | 84 | 55 | 11 | | 62 | 62 | | | |
| BBP-6 | HPCD 14% | 0 | | | | | 15 | | 58 | | | | |
| BBP-7 | PEI 0.05% | 0 | | | | | | | | | 150 | 122 | 135 |
| BBP-8 | PEI 0.1% | 0 | | | | | | | | | 150 | | |
| BBP-9 | PEI 0.1% No Ab | 0 | | | | | | | | | 0 | | |
| BBP-10 | NaCl 1M | 0 | 74 | 9 | 86 | 87 | 18 | | 97 | 78 | | | |
| BBP-11 | NaCl 2M | 0 | | | | | 21 | | 89 | 66 | | | |
| BBP-12 | Arg 0.5M | 0 | 59 | 12 | 85 | 74 | 1 | | 84 | 60 | | | |
| BBP-13 | Arg 1M | 0 | | | | | — | | 70 | 58 | | | |
| BBP-14 | 2M KP1, pH 7, | 0 | | | | | | | | | | | 26 |
| | 1M Tre | | | | | | | | | | | | |
| BBP-15 | 0.5M Tre | 0.5M Arg | | | | | 15 | | 81 | 53 | | | |
| BBP-16 | 4% HPCD | 0.5M Arg | | | | | 2 | | 81 | 49 | | | |
| BBP-17 | 0.05% PEI | 1M Arg | | | | | | | | | | | 93 |
| BBP-18 | 0.05% PEI | 0.05M | | | | | | | | | | | 137 |
| | | Arg/Glutamic | | | | | | | | | | | |
| | | acid | | | | | | | | | | | |
| BBP-19 | 1M NaCl | 0.5M Arg | | | | | 41 | 6 | 89 | 77 | | | |
| BBP-20 | 0.05 Arg | 0.05M Glutamic | | | | | | 12 | 100 | 62 | | | |
| | | acid | | | | | | | | | | | |
| BBP-21 | 1M Arg | 1M KPi | | | | | | | | | | | 57 |

TABLE 7
% Activity in High Temperature Stabilization Tests Rabbit Anti-Ricin Antibody

| | | | Run 1 | | | | Run 2 | | | | Run 3 | | Run 4 |
|---|---|---|---|---|---|---|---|---|---|---|---|---|---|
| | 1st | 2nd | 60° C. | | 55° C. | | 60° C. | | 55° C. | | 60° C. | | 60° C. |
| Sample # | Stabilizer | Stabilizer | D 1 | W 1 | W 1 | W 5 | W 1 | | W 1 | W 3 | W 1 | W 2 | W 1 |
| Control | 0 | 0 | 76 | 4 | 52 | 12 | | | | | 21 | 16 | 35 |
| | | 0 | | | | | | | | | | | |
| BB-RAR-1-1 | GA, 33% | 0 | 1 | — | — | — | | | | | | | |
| BB-RAR-1-2 | Gel A, 1% | 0 | | | | | | | | | 17 | 15 | |
| BB-RAR-1-3 | Gel A, 2% | 0 | 67 | 11 | 46 | 17 | | | | | 15 | 13 | |
| BB-RAR-1-4 | Gel A, 4% | 0 | | | | | | | | | 15 | 14 | |
| BB-RAR-1-5 | Gel B, 2% | 0 | 83 | 4 | 44 | 15 | | | | | | | |
| BB-RAR-1-6 | Tre 0.5M | 0 | 78 | 16 | 59 | 23 | 18 | | 53 | 21 | 17 | 9 | |
| BB-RAR-1-7 | Tre 1M | 0 | | | | | 27 | | 64 | 20 | 17 | 8 | |
| BB-RAR-1-8 | Tre 1.5M | 0 | | | | | | | | | 18 | 9 | 28 |
| BB-RAR-1-9 | T80 ® 0.01% | 0 | 88 | 5 | 54 | 14 | | | | | | | |
| BB-RAR-1-10 | T80 ® 0.1% | 0 | 107 | 10 | 54 | 10 | 9 | | 49 | 19 | | | |
| BB-RAR-1-11 | T80 ® 1% | 0 | | | | | 6 | 44 | 15 | | | | |
| BB-RAR-1-12 | NaCl, 1M | 0 | 20 | 6 | 42 | 33 | | | | | | | 36 |
| BB-RAR-1-13 | Arg, 1M | 0 | 24 | 1 | 36 | 17 | | | | | | | |
| BB-RAR-1-14 | 10% LiCl | 0 | | | | | 2 | | 42 | | | | |
| BB-RAR-1-15 | 2% Gel A | 0.5M Tre | | | | | 42 | | 80 | 33 | 24 | 21 | |
| BB-RAR-1-16 | 2% Gel A | 1M Tre | | | | | | | | | 29 | 23 | |
| BB-RAR-1-17 | 2% Gel A | 1.5M Tre | | | | | | | | | | | 37 |
| BB-RAR-1-18 | 2% Gel A | 1.5M Tre 50 mM | | | | | | | | | | | 39 |
| | | Arg/Glu | | | | | | | | | | | |
| BB-RAR-1-19 | 2% Gel A | 1.5M Tre 1M NaCl | | | | | | | | | | | 38 |
| BB-RAR-1-20 | 2% Gel A | 0.5M Tre 0.1m CaCl | | | | | | | | | 27 | 15 | |
| BB-RAR-1-21 | 2% Gel | 0.1% T80 ® | | | | | 12 | 80 | 30 | | | | |
| BB-RAR-1-22 | 2% Gel A | 1M NaCl | | | | | | | | | 9 | | |
| BB-RAR-1-23 | 10% Gel A | 10% LiCl | | | | | 3 | | 54 | | | | |
| BB-RAR-1-24 | 75 mM Arg | 5% glycerol | | | | | 16 | | 64 | 36 | | | |
| | 75 mM Glu | | | | | | | | | | | | |

TABLE 8
% Activity in High Temperature Stabilization Tests the Rabbit Anti-Ovalbumin Antibody
Sample BB-RAR-2

| | | | Run 1 | | | | Run 2 | | | | Run 3 | | Run 4 |
|---|---|---|---|---|---|---|---|---|---|---|---|---|---|
| | 1st | 2nd | 60° C. | | 55° C. | | 60° C. | | 55° C. | | 60° C. | | 60° C. |
| Sample # | Stabilizer | Stabilizer | D 1 | W 1 | W 1 | W 5 | W 1 | | W 1 | W 3 | W 1 | W 2 | W 1 |
| Control | 0 | 0 | 66 | 34 | 62 | | 11 | | 16 | 0 | 12 | 4 | 42 |
| BB-RAR-2 | Gel B, 1% | 0 | | | | | | | | | 29 | | |
| | Gel B, 2% | 0 | 74 | 39 | 90 | | | | | | 24 | 24 | |
| | Gel B, 4% | 0 | | | | | | | | | 29 | 20 | |
| | Tre 0.5M | 0 | 72 | 34 | 63 | | | | | | 13 | 4 | |
| | Tre, 1M | 0 | | | | | 20 | | 20 | 5 | 20 | 7 | |
| | Tre 1.5M | 0 | | | | | | | | | 16 | 12 | |
| | HPCD 4% | 0 | 72 | 29 | 60 | | | | | | | | |
| | HPCD 8% | 0 | | | | | 15 | | 10 | 2 | | | |
| | T80, 1% | 0 | | | | | 9 | | 9 | 1 | | | |
| | T80 .1% | 0 | 77 | 35 | 65 | | | | | | | | |
| | T80 .01% | 0 | 75 | 34 | 63 | | | | | | | | |
| | Arg 1M | 0 | 65 | 26 | 70 | | | | | | | | |
| | 1M KPi | 0 | | | | | | | | | 11 | 2 | |
| | 2M KPi | 0 | | | | | 51 | | 63 | 17 | 34 | 31 | 57 |
| | 3M KPi | 0 | | | | | | | | | 36 | 31 | |
| | 0.1M CaCl₂ | 0 | | | | | | | | | 0 | 0 | |
| | HEPES pH7 | 0 | | | | | | | | | 21 | 15 | 0 |
| | 20% LiCl | 0 | | | | | 3 | | 2 | | | | |
| | 0 | 2M KPi 0.5M Tre | | | | | | | | | 16 | 3 | |
| | 0 | 2M KPi 1.0M Tre | | | | | | | | | 18 | 1 | |
| | 0 | 2M KPi 1.5M Tre | | | | | | | | | 37 | 0 | |
| | 2% Gel B | 0.5M Tre | | | | | 36 | | 40 | 40 ± 5 | 31 | 42 | |
| | 2% Gel B | 1.0M Tre | | | | | | | | | 28 | 34 | |
| | 2% Gel B | 1.5M Tre | | | | | | | | | 32 | 47 | 56 |
| | 2% Gel B | 1.5M Tre 10M Arg | | | | | | | | | | | 10 |
| BB-RAR-2 | 2% Gel B | 1.5M Tre 20 mM HEPES | | | | | | | | | | | |
| | 2% Gel B | 0.5M Tre 0.1M CaCl₂ | | | | | | | | | 32 | 32 | |
| | 2% Gel B | 0.1% T80 | | | | | 30 | 47 | 16 | | | | |
| | 2% Gel B | 4% HPCD | | | | | 23 | | 38 | | | | |
| | 1M Arg | Pi | | | | | | | | | | | 44 |
| | 75 mM Arg | 75 mM Glu 5% glycerol | | | | | 20 | | 34 | 8 | | | |

TABLE 9
% Activity in Anti-Yp Monoclonal Antibody Stability

| | | | 55° C. | | | | 60° C. |
|---|---|---|---|---|---|---|---|
| | | | Activity Recovery | | | | Activity Recovery |
| Sample # | 1^{st} Stabilizer | 2^{nd} Stabilizer | 1 W | 2 W | 4 W | 8 W | 1 Day |
| Control | 0 | 0 | 122 ± ±9 | 37 ± 8 | 4 ± 1 | ND | 1 ± 1 |
| CC1 | 0 | 1 M Trehalose | 156 ± 69 | 56 ± 13 | 24 ± 11 | 6 ± 2 | 42 ± 23 |
| CC2 | 0.5 M Arg•HCl | 0 | 110 ± 26 | 28 ± 11 | 3 ± 1 | ND | 0 ± 0 |
| CC3 | 0.05 M ARG/Glu | 0 | >89 | 43 ± 9 | 48 ± 13 | 1 ± 1 | 5 ± 9 |
| CC4 | 0.05M Arg/Glu | 1M Trehalose/ | >98 | 52 ± 13 | 48 ± 34 | 5 ± 1 | 81 ± 22 |
| CC5 | 0.05M Arg/Glu | 1 M KPi | >91 | 41 ± 11 | 76 ± 29 | 3 ± 1 | 88 ± 11 |

W = Week

### Materials and Methods

The following methods and materials were used to prepare the antibodies used in the tests described in Tables 5-9 above.

TABLE D
Antibody Stabilization
Antibodies

| | |
|---|---|
| Anti-Yp Monoclonal | A monoclonal antibody to the |
| | F1 antigen of Yp was obtained |
| | from Biodesign (C86308M) |
| Goat Anti-Yp | Goat polyclonal antibodies to |
| Polyclonal | the Fl antigen of Yp were |
| | produced internally at Battelle. |
| | The IgG fraction was prepared by |
| | Protein G chromatography |
| Rabbit Anti-Ricin | Goat polyclonal antibodies to |
| | autoclaved ricin were produced |
| | internally at Battelle. The IgG |
| | fraction was prepared by Protein |
| | G chromatography. |
| Rabbit Anti-Ovalbumin | An IgG fraction of rabbit polyclonal |
| | antibodies to ovalbumin was obtained |
| | from Research Diagnostics, Inc. |

Antigens

| | |
|---|---|
| F1 antigen of Yp | The F1 antigen was prepared at |
| | Battelle by the method of |
| | Andrews et. al. (1996), Immunity, |
| | 64, 2180-2187 |
| Ricin | Ricin was purchased from Vector |
| | Laboratories as a heat inactivated |
| | preparation. The preparation was |
| | autoclaved at Battelle for 15 minutes |
| | and sonicated to re-solubilize any |
| | precipitated protein. |
| Ovalbumin | Purchased from Sigma |

### Indirect ELISA Assay Method

### Plate Coating

The antigen is diluted in coating buffer and 0.1 ml aliquots are added to the required number of wells on a 96-well polystyrene microtiter plate. The coating buffers and antibody concentrations for the four antigens are shown in Table E below:

TABLE E
Coating Buffers and Antibody Concentrations

| | Coating | | |
|---|---|---|---|
| Antigen | Concentration | | Coating Buffer |
| F1 (Yp) | 10 | μg/ml | 0.05 M Carbonate buffer, |
| | | | pH 9.4 |
| Ricin | 1 | μg/ml | 0.05 M Carbonate buffer, |
| | | | pH 9.4 |
| Ovalbumin | l | μg/ml | Phosphate Buffered Saline |
| | | | (PBS, pH 7.4) |

The plates are incubated either at 4° C. overnight or at 37° C. for 90 minutes to coat the plate with antigen. Plates coated at 4° C. were stored for up to five days before use. Plates coated at 37° C. were used the same day.

### Plate Wash

The plates can be washed either by hand or with an automatic device. The liquid in the wells is removed by aspiration and 0.3 ml of wash buffer (0.01 M phosphate buffer, pH 7.4, 0.05% Tween 20) is added via a manual or automated pipet. The sequence of aspiration and buffer addition is repeated two more times. Finally the aspirated plate is inverted and tapped forcefully several times on paper towels to remove the remaining liquid from the wells.

### Plate Block

Each well is filled with 0.25-0.3 ml, of blocking buffer (0.01 M phosphate buffer, pH 7, 0.5% bovine serum albumin), covered, and incubated at room temperature for 90 minutes. The plate is then washed using the plate wash procedure.

### Sample and Standards Load

The antibody samples to be assayed are diluted appropriately in dilution buffer (0.01 M phosphate buffer, pH 7.4, 0.05% Tween 20, 0.5% bovine serum albumin) and 0.1 ml, aliquots are added to the wells via a pipet. The antibody standards are similarly diluted and added to the wells. The plates are then covered, incubated at room temperature for 90 minutes, and then washed using the plate wash procedure. The antibody concentrations used for the standard curves is shown In Table F below:

TABLE F
Antibody Concentrations for Std. Curve.

| | |
|---|---|
| Antibody | Standard Curve Concentrations |

| | | |
|---|---|---|
| Anti-Yp Monoclonal | 1 μg/ml −> | 0.001 μg/ml, |
| | | dilute by factor of three |
| Goat Anti-Yp | 20 μg/ml −> | 0.313 μg/ml, |
| Polyclonal | | dilute by factor of two |
| Rabbit Anti-Ricin | 10 μg/ml −> | 0.078 μg/ml, |
| | | dilute by factor of two |
| Rabbit Anti-Ovalbumin | 12 μg/ml −> | 0.047 μg/ml, |
| | | dilute by factor of two |

### Antibody Detection Reagent Addition

Protein detection reagent is added in 0.1 ml aliquots to each well and the plates are covered and incubated for 90 minutes at room temperature. The protein detection reagents for the four antibodies are shown in Table G below:

TABLE G
Protein Detection Reagents

| | Standard Curve |
|---|---|
| Antibody | Concentrations |
| Anti-Yp Monoclonal | Anti-Mouse IgG antibody |
| | conjugated to Horse |
| | Radish Peroxidase |
| Goat Anti-Yp | Protein G conjugated to |
| Polyclonal | |
| Rabbit Anti-Ricin | Protein G conjugated to |
| | Horse Radish Peroxidase |
| Rabbit Anti-Ovalbumin | Protein A conjugated to |
| | Horse Radish Peroxidase |

The plate is then washed using the plate wash procedure.

### Substrate Addition

The peroxidase substrate, prepared by mixing one part ABTS A and one part ABTS B, is added, in 0.1 ml, aliquots to each well. The plate is then covered and incubated in the dark for 30 minutes.

### Plate Assay

The absorbance of each well is read in a Molecular Devices microtiter plate reader at 420 nm. The standard curve is generated using a four-parameter curve fit via the Soft-Max software and the samples are quantitated from the standard curve.

### Stabilization Studies

### Stock Antibody Solutions

The stock solutions of antibodies are diluted to 0.1 mg/ml in the various formulation buffers. The stock antibody solutions are shown below:

| | Standard Curve |
|---|---|
| Antibody | Concentrations |
| Anti-Yp Monoclonal | 3.8 mg/ml solution in PBS, |
| | stored at 4° C. |
| Goat Anti-Yp | 2.0 mg/ml solution in PBS, |
| Polyclonal | stored frozen |
| Rabbit Anti-Ricin | 8 mg/ml solution in PBS |
| | stored at 4° C. |
| Rabbit Anti-Ovalbumin | Stored as a lyophilized powder. |
| | Before use, dissolve in deionized |
| | water at l mg protein/ml. This |
| | solution contains 0.002 M potassium |
| | phosphate buffer, pH 7.2 and |
| | 0.01 15 M NaCl |
| Incubation Conditions | Antibody samples are incubated as |
| | 0.1 ml aliquots in 0.3 ml polypropylene |
| | tubes with screw caps and O-ring seals. |
| | The samples are stored in incubators at |
| | the various temperatures. The entire |
| | content of each tube is used for the assay. |

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the spirit of the scope of the invention.

What is claimed:

1. A method for preparing a temperature stabilized solution or gel of a biologically active protein in need of stabilization which comprises combining said protein to be stabilized with a stabilizing effective amount of a first stabilizer and a stabilizing effective amount of a second stabilizer;
wherein said first stabilizer is selected from the group consisting of:
a) an amino acid;
b) a peptide;
c) a polypeptide; and
d) a poly(amino acid); and
wherein said second stabilizer is selected from the group consisting of:
a) a surfactant;
b) a monosaccharide
c) a disaccharide;
d) an inorganic salt;
e) ectoine;
f) a polyionic compound; and
g) an amino acid, peptide, polypeptide or poly(amino acid), provided that such amino acid, peptide, polypeptide, or poly(amino acid) is not selected as said first stabilizer; and
h) combinations of any of components a-g of said second stabilizer group.

2. The method according to claim 1 wherein said protein is selected from the group consisting of polymerases, restriction enzymes, antibodies, diagnostic proteins and therapeutic proteins.

3. The method according to claim 2 wherein said protein is a polymerase.

4. The method according to claim 3 wherein said polymerase is Taq polymerase.

5. The method according to claim 2 wherein said protein is a restriction enzyme.

6. The method according to claim 5 wherein said restriction enzyme is selected from the group consisting of Ava I, Bam HI, BgI II, Eco RI, Eco RII, Eco RV, Hae III, Hha I, Hind III, Hpa I, Kpn I, Mbo I, Pst I, Sma I, Sstl, Sal I, Taq I, and Xma I.

7. The method according to claim 2 wherein said protein is an antibody.

8. The method according to claim 7 wherein said antibody is selected from the group consisting of anti-Yp monoclonal antibody, goat anti-Yp polyclonal antibody, rabbit anti-ricin antibody, and rabbit anti-ovalbumin antibody.

9. The method according to claim 2 wherein said diagnostic protein is selected from the group consisting of the 31 kD protein from Schistosoma mansoni worms; purified protein from M. paratuberculosis, polyamine-modified Aβ40, and A-Protein.

10. The method according to claim 2 wherein said therapeutic protein is selected form the group consisting of peginterferon alpha 2-a, adalimumab, agalsidase beta, alfacet, trastuzumab, darbepoetin, infliximab, rituxamab, tositumomab, bevacizumab, and cetuximab.

11. The method according to claim 1 wherein said first stabilizer is an amino acid, a poly(amino acid) and mixtures thereof.

12. The method according to claim 11 wherein said amino acid is arginine or glutamic acid and mixtures thereof.

13. The method according to claim 11 wherein said poly(amino acid) is poly(glutamic acid).

14. The method according to claim 1 wherein said is first stabilizer is selected from the group consisting of peptides and polypeptides and mixtures thereof.

15. The method according to claim 1, wherein said second stabilizer is selected from the group consisting of a surfactant, a monosaccharide, a disaccharide, an inorganic salt, ectoine and polyionic compounds and mixtures thereof.

16. The method according to claim 1, wherein said second stabilizer is selected from the group consisting of an amino acid, peptide, polypeptide or poly(amino acid), provided that such amino acid, peptide, polypeptide, or poly(amino acid) is not selected as said first stabilizer.

17. The method according to claim 15, wherein said second stabilizer is selected from the group consisting of a surfactant, ectoine, and a polyionic compound or mixtures thereof.

18. The method according to claim 17, wherein said second stabilizer is a surfactant selected from the group consisting of dipalmitoylphosphatidylcholine, polyoxyethylene (20) sorbitan monooleate, polyoxyethylene (20) sorbitan monolaurate, polyoxyethylene-polyoxypropylene block copolymer, polyoxyethyleneglycol dodecyl ether, gelatin and glycerol or mixtures thereof.

19. The method according to claim 15, wherein said second stabilizer is an inorganic salt selected from the group consisting of NaCl, MgCl₂, KCl, K₂SO₄, Na₂SO₄, Na₃PO₄, and K₃PO₄ or mixtures thereof.

20. The method according to claim 15, wherein said second stabilizer is selected from the group consisting of monosaccharides and disaccharides and mixtures thereof.

21. The method according to claim 20, wherein said second stabilizer is selected from the group consisting of lactose, maltose, melibiose, sucrose, and trehalose and mixtures thereof.

22. The method according to claim 21, wherein said second stabilizer is trehalose.

23. The method according to claim 17, wherein said second stabilizer is ectoine.

24. The method according to claim 17 wherein said polyionic compound is selected from the group consisting of polyethleneimine and polyacrylic acid.

25. The method according to claim 1 wherein said protein is a polymerase and wherein said first stabilizer is arginine and wherein said second stabilizer is selected from the group consisting of ectoine and trehalose and mixtures thereof.

26. The method according to claim 2 wherein said solution or gel is an aqueous solution or gel.

27. The method according to claim 25 wherein said polymerase is Taq polymerase.

28. The method according to claim 2 wherein said protein is a polymerase; wherein said first stabilizer is arginine; wherein said second stabilizer is a surfactant selected from the group consisting of dipalmitoylphosphatidylcholine, polyoxyethylene (20) sorbitan monooleate, polyoxyethylene (20) sorbitan monolaurate, polyoxyethylene-polyoxypropylene block copolymer, and polyoxyethyleneglycol dodecyl ether; and wherein said solution or gel is an aqueous solution or gel.

29. A formulation containing a temperature stabilized solution or gel of a biologically active protein in need of stabilization which formulation comprises a combination of said protein and a stabilizing effective amount of a first stabilizer and a stabilizing effective amount of a second stabilizer;
wherein said first stabilizer is selected from the group consisting of:
a) an amino acid;
b) a peptide;
c) a polypeptide; and
d) a poly(amino acid); and
wherein said second stabilizer is selected from the group consisting of:
a) a surfactant;
b) a monosaccharide
c) a disaccharide;
d) an inorganic salt;
e) ectoine;
f) a polyionic compound; and
g) an amino acid, peptide, polypeptide or poly(amino acid), provided that such amino acid, peptide, polypeptide, or poly(amino acid) is not selected as said first stabilizer; and
h) combinations of any of components a-g of said second stabilizer group.

30. The formulation according to claim 29 wherein said protein is selected from the group consisting of polymerases, restriction enzymes, antibodies, diagnostic proteins and therapeutic proteins.

31. The formulation according to claim 30 wherein said solution or gel is an aqueous solution or gel.

32. The formulation according to claim 30 wherein said protein is a polymerase.

33. The formulation according to claim 32 wherein said polymerase is Taq polymerase.

34. The formulation according to claim 30 wherein said protein is an antibody.

35. The formulation according to claim 34 wherein said antibody is selected from the group consisting of anti-Yp monoclonal antibody, goat anti-Yp polyclonal antibody, rabbit anti-ricin antibody, and rabbit anti-ovalbumin antibody.

36. The formulation according to claim 30 wherein said protein is a restriction enzyme.

37. The formulation according to claim 36 wherein said restriction enzyme is selected from the group consisting of Ava I, Bam HI, BgI II, Eco RI, Eco RII, Eco RV, Hae III, Hha I, Hind III, Hpa I, Kpn I, Mbo I, Pst I, Sma I, SstI, Sal I, Taq I, and Xma I.

38. The formulation according to claim 30 wherein said diagnostic protein is selected from the group consisting of the 31 kD protein from Schistosoma mansoni worms; purified protein from M. paratuberculosis, polyamine-modified Aβ40, and A-Protein.

39. The formulation according to claim 30 wherein said therapeutic protein is selected from the group consisting of peginterferon alpha 2-a, adalimumab, agalsidase beta, alfacet, trastuzumab, darbepoetin, infliximab, rituxamab, tositumomab, bevacizumab, and cetuximab.

40. The formulation according to claim 29 wherein said first stabilizer is an amino acid, a poly(amino acid) and mixtures thereof.

41. The formulation according to claim 40 wherein said amino acid is selected from the group consisting of arginine and glutamic acid or mixtures thereof.

42. The formulation according to claim 40 wherein said poly(amino acid) is poly(glutamic acid).

43. The formulation according to claim 29 wherein said is first stabilizer is selected from the group consisting of peptides and polypeptides and mixtures thereof.

44. The formulation according to claim 29, wherein said second stabilizer is selected from the group consisting of a surfactant, a monosaccharide, a disaccharide, an inorganic salt, ectoine and polyionic compounds and mixtures thereof.

45. The formulation according to claim 29, wherein said second stabilizer is selected from the group consisting of an amino acid, peptide, polypeptide or poly(amino acid) and mixtures thereof, provided that such amino acid, peptide, polypeptide, or poly(amino acid) is not selected as said first stabilizer.

46. The formulation according to claim 44, wherein said second stabilizer is selected from the group consisting of a surfactant, ectoine, a polyionic compound and mixtures thereof.

47. The formulation according to claim 46, wherein said second stabilizer is a surfactant selected from the group consisting of dipalmitoylphosphatidylcholine, polyoxyethylene (20) sorbitan monooleate, polyoxyethylene (20) sorbitan monolaurate, polyoxyethylene-polyoxypropylene block copolymer, polyoxyethyleneglycol dodecyl ether, gelatin and glycerol and mixtures thereof.

48. The formulation according to claim 44, wherein said second stabilizer is an inorganic salt selected from the group consisting of NaCl, MgCl₂, KCl, K₂SO₄, Na₂SO₄, Na₃PO₄, and K₃PO₄ or mixtures thereof.

49. The formulation according to claim 44, wherein said second stabilizer is selected from the group consisting of monosaccharides and disaccharides and mixtures thereof.

50. The formulation according to claim 49, wherein said second stabilizer is selected from the group consisting of lactose, maltose, melibiose, sucrose, and trehalose and mixtures thereof.

51. The formulation according to claim 50, wherein said second stabilizer is trehalose.

52. The formulation according to claim 44, wherein said second stabilizer is ectoine.

53. The formulation according to claim 44 wherein said polyionic compound is selected from the group consisting of polyethleneimine and polyacrylic acid.

54. The formulation according to claim 29 wherein said protein is a polymerase; wherein said first stabilizer is arginine; and wherein said second stabilizer is selected from the group consisting of ectoine and trehalose and mixtures thereof.

55. The formulation according to claim 54 wherein said polymerase is Taq polymerase.

56. The formulation according to claim 31 wherein said protein is a polymerase; wherein said first stabilizer is arginine; and wherein said second stabilizer is a surfactant selected from the group consisting of dipalmitoylphosphatidy(choline, polyoxyethylene (20) sorbitan monooleate, polyoxyethylene (20) sorbitan monolaurate, polyoxyethylene-polyoxypropylene block copolymer, and polyoxyethyleneglycol dodecyl ether.

57. A method for preparing a temperature stabilized aqueous solution or gel of Taq polymerase which comprises combining said Taq polymerase with:
(i) a stabilizing effective amount of a first stabilizer, wherein said first stabilizer is selected from the group consisting of:
a) a basic amino acid;
b) an acidic amino acid;
c) an acidic or a basic poly(amino acid); and
(ii) a second stabilizer selected from the group consisting of:
a) a surfactant;
b) a monosaccharide or a disaccharide;
c) one or more of an inorganic salt;
d) ectoine;
e) combinations of any of components a)-c) of said first stabilizer group provided that such basic amino acid, acidic amino acid, or said acidic or basic poly(amino acid) is not selected as said first stabilizer; and
f) combinations of any of components a)-e) of said second stabilizer group.

58. The method according to claim 57 wherein said first stabilizer is arginine.

59. The method according to claim 57 wherein said first stabilizer is poly(glutamic acid).

60. The method according to claim 57 wherein said second stabilizer is a surfactant selected from the group consisting of dipalmitoylphosphatidylcholine, polyoxyethylene (20) sorbitan monooleate, polyoxyethylene (20) sorbitan monolaurate, polyoxyethylene-polyoxypropylene block copolymer, polyoxyethyleneglycol dodecyl ether, gelatin and glycerol or mixtures thereof.

61. The method according to claim 57 wherein said second stabilizer is ectoine.

62. A temperature stabilized aqueous solution or gel formulation of Taq polymerase which comprises combining said Taq polymerase with:
(i) a stabilizing effective amount of a first stabilizer, wherein said first stabilizer is selected from the group consisting of:
a) a basic amino acid;
b) an acidic amino acid;
c) an acidic or a basic poly(amino acid); and
(ii) a second stabilizer selected from the group consisting of:
a) a surfactant;
b) a monosaccharide or a disaccharide;
c) one or more of an inorganic salt;
d) ectoine;
e) combinations of any of components a)-c) of said first stabilizer group provided that such basic amino acid, acidic amino acid, or said acidic or basic poly(amino acid) is not selected as said first stabilizer; and
f) combinations of any of components a)-e) of said second stabilizer group.

63. The formulation according to claim 62 wherein said first stabilizer is arginine.

64. The formulation according to claim 62 wherein said first stabilizer is poly(glutamic acid).

65. The method according to claim 62 wherein said second stabilizer is a surfactant selected from the group consisting of dipalmitoylphosphatidylcholine, polyoxyethylene (20) sorbitan monooleate, polyoxyethylene (20) sorbitan monolaurate, polyoxyethylene-polyoxypropylene block copolymer, polyoxyethyleneglycol dodecyl ether, gelatin and glycerol or mixtures thereof.

66. The method according to claim 62 wherein said second stabilizer is ectoine.
